(19) United States
(12) Patent Application Publication
(10) Pub. No.: US 2008/0071149 A1
(43) Pub. Date: Mar. 20, 2008

(54) METHOD AND SYSTEM OF REPRESENTING A MEDICAL EVENT

(76) Inventor: Collin Rich, Ypsilanti, MI (US)

Correspondence Address: SCHOX PLC, 209 N. MAIN STREET #200, ANN ARBOR, MI, 48104, US

(21) Appl. No.: 11/858,806

(22) Filed: Sep. 20, 2007

Related U.S. Application Data
(60) Provisional application No. 60/826,330, filed on Sep. 20, 2006.

(51) Int. Cl. A61B 5/00

(52) U.S. Cl. 600/300

(57) ABSTRACT

The present invention includes a system and a method of representing a medical event. The method includes the steps of collecting 3D information on an event, identifying a non-linear aspect of the event, determining a non-planar slice of the 3D information that represents the non-linear aspect, and outputting the non-planar slice as a representation of the event. The system includes an imaging device for collecting 3D information on an event. The system further includes a processor for identifying a non-linear aspect of the event, determining a non-planar slice of the 3D information that represents the non-linear aspect and outputting the non-planar slice as a representation of the event.

### CROSS-REFERENCE TO RELATED APPLICATIONS

This application claims the benefit of U.S. Provisional Application No. 60/826,330 filed 20 Sep. 2006 and entitled “Method and System of Representing a Medical Event”, which is incorporated in its entirety by this reference.

### TECHNICAL FIELD

This invention relates generally to the medical field, and more specifically to a new and useful method of representing a medical event in the field of medical imaging.

### BACKGROUND

It is common in medical practice to use an imaging device to guide the use of medical devices. For example, ultrasound devices are often used to guide the insertion of a biopsy needle. The imaging device typically provides a two dimensional slice of the patient's anatomy and the medical device. The use of the imaging device, however, requires great skill because even a small translation or rotation of the imaging plane from the axis of the medical device leaves the medical device out of view. Furthermore, many medical devices include long, thin needles that can bend out-of-plane during the insertion process, making it impossible to observe a large portion of the device with a slice image. Additionally, the device and target location may not be in plane during the procedure, making it difficult to assess whether the trajectory of the device is on-target.

Thus, there is a need in the medical imaging field to create a new and useful method of representing a medical event. The present invention provides such new and useful method, along with an accompanying system for representing a medical event.

### BRIEF DESCRIPTION OF THE FIGURES

FIG. 1 is schematic diagram of the preferred system for representing a medical event and a non-planar slice of the medical event.

FIG. 2 is schematic diagram of the prior art system for representing a medical event, and a planar slice of the medical event.

FIG. 3 is schematic diagram of the preferred system for representing a medical event, and two non-planar slices of the medical event.

FIG. 4 is a flow chart depicting a preferred method of representing a medical event.

### DESCRIPTION OF THE PREFERRED EMBODIMENT

The following description of the preferred embodiments of the invention is not intended to limit the invention to these preferred embodiments, but rather to enable any person skilled in the art of medical imaging to make and use this invention.

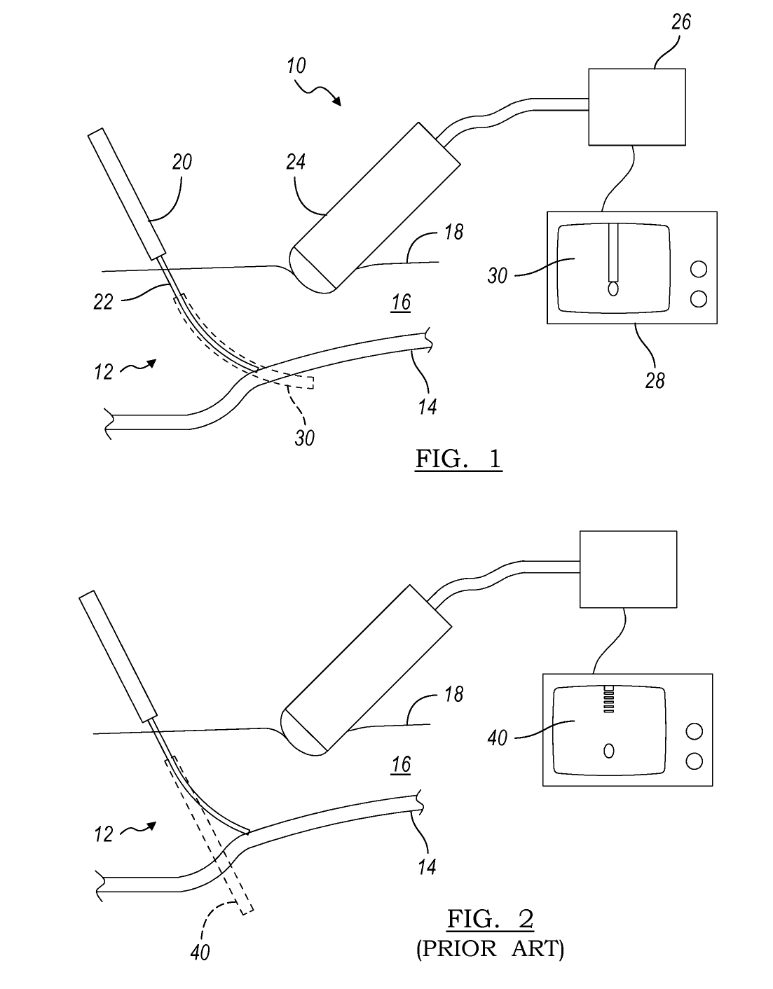

As shown in FIG. 1, the preferred embodiment of the present invention includes a system 10 and a method of representing a medical event, such as the insertion of a biopsy needle into a body 12. The system and method are preferably used to guide the use of medical instrument 20 to analyze or treat the body 12. The system preferably includes an imaging device 24 to collect three-dimensional information on the medical event, a processor 26 to process the collected 3D information, to identify one or more non-linear aspects of the medical event, and to determine at least one non-planar slice of the three-dimensional information that represents or enhances the non-linear aspect of the medical event, and a display 28 to render the non-planar slice(s) into a visual format that can be used by an operator during the medical event. The system and method may, however, be used to represent any suitable event, especially in an event that requires precision targeting of an object within a body.

The method includes the steps of collecting 3D information on an event, identifying a non-linear aspect of the event, determining a non-planar slice of the 3D information that represents the non-linear aspect, and outputting the non-planar slice as a representation of the event. The system includes an imaging device for collecting 3D information on an event. The system further includes a processor for identifying a non-linear aspect of the event, determining a non-planar slice of the 3D information that represents the non-linear aspect and outputting the non-planar slice as a representation of the event.

The body 12, which is not an element of the preferred system 10, may be a human body, an animal body, or any other suitable body. The body 12 may include a vessel 14 such as an artery, vein, or other similar structure for carrying fluids, into which one may extract or inject additional fluids, such as radioisotopes, with the medical instrument. The vessel 14 may be located within a segment of tissue 16, such as adipose or muscle tissue. A surface 18, such as an epidermis, may bound the body 12 and contain the vessel 14 and the tissue 16.

The medical instrument 20 of the preferred embodiment functions to analyze or treat the body 12. The medical instrument 20 preferably includes a needle or other projecting portion for penetrating the surface 18 of the body 12, intersecting with the vessel 14, and either injecting fluids into the vessel 14 or extracting fluids from the vessel 14. The medical instrument 20 is preferably handled by an operator, such as a physician, nurse, or emergency medical technician. The medical instrument may, however, be any suitable device to analyze or treat the body 12. The medical instrument 20 preferably includes one or more markers 22. The markers 22 function to provide information regarding the position of the medical instrument 20. The markers 22 are preferably acoustic, electromagnetic, or radiological elements that provide information regarding the position of the medical instrument 20. The markers 20 preferably provide signals or reflections of signals that are transmitted through the body 12 and receivable by the imaging device 24 of the system 10. Alternatively, the medical instrument 20 may be fully or partially coated with a coating (not shown) that is selected for its unique acoustic, electromagnetic, or radiological properties for providing information regarding the position of the medical instrument 20. The medical instrument may, however, include any device or method to provide information regarding the position of the medical instrument 20.

The imaging device 24 of the preferred embodiment functions to collect three-dimensional information on a medical event. The collected 3D information preferably includes the position and trajectory of the medical instrument 20 as well as the position of any vessels 14 within the body and the trajectory of any fluids, such as blood, flowing in the vessels. The imaging device 24 is preferably an ultrasound system that is capable of collecting three-dimensional information on a medical event. The imaging device 24 may alternatively include MRI devices, CT devices, PET devices, or any other suitable device for collecting three-dimensional information on a medical event.

The processor 26 of the preferred embodiment functions to receive and process the collected 3D information from the imaging device 24. The processor 26 may be a distinct or integral component of the imaging device 24. The processor 26 may also be coupled to an interface (not shown) to permit an operator to select and identify medical targets. Alternatively, the processor may include an additional device to communicate directly with the markers 22 disposed on the medical instrument 20, thereby directly providing information regarding the position and trajectory of the medical instrument 20.

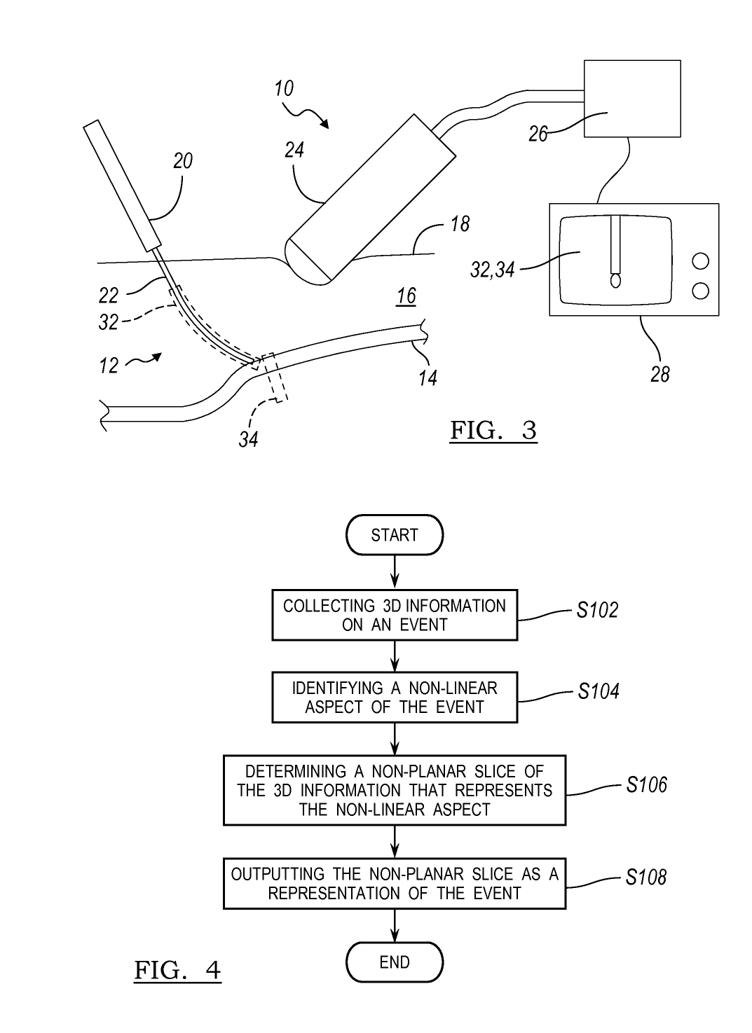

As shown in FIGS. 1 and 3, the processor 26 of the preferred embodiment further functions to process the collected information and identify a non-linear aspect of the medical event (shown in FIG. 1) or more than one non-linear aspects of the medical event (shown in FIG. 3). The processor 26 preferably includes software to accomplish this function, but may alternatively include integrated hardware. The non-linear aspects of the medical event may include a non-linear portion of the medical instrument 20, which may be identified by the markers 22 on the medical instrument 20 or any other suitable method or device. The non-linear aspects of the medical event may also include a non-linear trajectory of the medical instrument or of blood flow, which may be identified by pattern recognition and segmentation techniques performed by the processor, by time-based pattern recognition techniques performed by the processor 26, through user selection of a segment of the vessel, or any other suitable method or device.

The processor 26 of the preferred embodiment further functions to determine at least one non-planar slice of the three-dimensional information that represents or enhances the non-linear aspect of the medical event. The determination of the non-planar slice includes defining at least one non-linear line that coincides with the non-linear aspect of the medical event, and extrapolating from the non-linear line a non-planar slice that extends in at least one additional dimension from the non-linear line. One or more non-planar slices can be determined for each non-linear aspect of the medical event. The first non-planar slice 32 and the second non-planar slice 34 do not necessarily share a continuous plane (shown in FIG. 3), in which the second non-planar slice 34 is slightly elevated relative to the first non-planar slice 32. The non-linear aspect is represented or enhanced when a non-linear slice allows viewing of at least a larger portion of the non-linear aspect, as best shown by a comparison of the system 10 and the non-planar slice 30 (shown in FIG. 1) of the preferred embodiment and the system and the planar slice 40 (shown in FIG. 2) of the prior art.

As shown in FIG. 1, the display 28 of the preferred embodiment, which is preferably connected to the processor 26, functions to render the non-planar slice(s) into a visual format that can be used by an operator during the medical event. The display 28 preferably includes a CRT, LCD, plasma screen, or any other suitable device that is capable of rendering the data received from the processor 26 into a visual format. The display 28 is preferably sized for ease of use and maneuverability within a medical facility such as an operating room, emergency room, or emergency vehicle.

As shown in FIG. 4, the preferred method for representing a medical event includes four steps. Step S102 of the method includes collecting three-dimensional information on an event. Step S104 of the method includes identifying a non-linear aspect of the event. Step S106 of the method includes determining a non-planar slice of the three-dimensional information that represents or enhances the non-linear aspect. Finally, Step S108 includes outputting the non-planar slice as a representation of the event. The method is preferably performed in a medical imaging application, such as those requiring the precise introduction of a medical instrument into a body. Alternatively, the method is usable in any application that requires the precise targeting of a first element relative to a second element, wherein the first element is contained within a body. The method is preferably performed with the system 10, but may be performed with any suitable system.

Step S102 of the method includes collecting three-dimensional information on a medical event. Step S102 is preferably performed by the imaging device 24, but may be performed by any suitable device. More preferably, step S102 includes generating acoustic waves and receiving acoustic echoes using a three-dimensional ultrasound system. As is known in the art, ultrasound systems are well suited for discriminating between structures having different acoustic properties, and especially structures that include a fluid flow. Owing to the Doppler effect, an ultrasound system will receive acoustic echoes that are distinct for vessels that include a fluid flow, as the motion of the fluid causes the frequency of the acoustic waves to be red-shifted by a known amount. As such, a preferred ultrasound system provides three-dimensional data distinguishing between different types of tissues and instruments in a medical event.

Step S104 of the method includes identifying a non-linear aspect of the event. This step is preferably performed by the processor 26, but may be performed by any suitable device. Step S104 of the method is preferably conducted by software that is usable in the performance of this function. Alternatively, step S104 may be performed by integrated hardware disposed within the processor 26 that identifies the one or more non-linear aspects of the medical event. Alternatively, step S104 may be performed on multiple non-linear aspects of the event simultaneously or substantially simultaneously.

The one or more non-linear aspects of the medical event include a non-linear portion of the medical instrument 20, which may be identified by the markers 22 or a coating on the medical instrument 20. The one or more non-linear aspects of the medical event may also include a non-linear trajectory of the medical instrument 20 or of blood flow through the vessel 14, which may be identified by time-based pattern recognition techniques performed by software operated by the processor 26. The one or more non-linear aspects of the medical event may also include a non-linear aspect of a medical target, such as a vessel 14 within a body 12, which is identified by pattern recognition and segmentation techniques preferably performed by software operated by the processor 26. Alternatively, the non-linear aspect of the medical target may be identified through user selection of a segment of the vessel, which is preferably received through the user interface coupled to the processor 26 and provided according to the preferred method.

Step S106 of the method includes determining a non-planar slice of the three-dimensional information that represents or enhances the non-linear aspect of the event. Following step S104, in which the one or more non-linear aspects are identified, step S106 functions to process the data identifying the one or more non-linear aspects of the medical event. Step 106 is preferably performed by the processor 26, which preferably includes software that is operable in the accomplishment of this function, but may be preformed by any suitable device or method. The determination of the non-planar slice includes the further steps of: defining at least one non-linear line that coincides with the at least one non-linear aspect of the medical event, and extrapolating from the at least one non-linear line a non-planar slice that extends in at least one additional dimension from the non-linear line. One or more non-planar slices can be determined for each non-linear aspect of the medical event.

Step S108 of the method includes outputting the one or more non-planar slices as a representation of the event. Step S108 of the method preferably includes the additional step of visually displaying the one or more non-planar slices. This step enables an operator of the medical instrument 20 to better understand the spatial relationship between the medical instrument 20 and the vessel 14. Step S108 is preferably performed by the display 28 which functions to render the one or more non-planar slices into a two dimensional visual format usable by an operator in directing the medical instrument 20 to the vessel 14, but may be performed by any suitable device. Preferably, the display 28 is sized for ease of use and maneuverability within a medical facility such as an operating room, emergency room, or emergency vehicle such that the preferred method may be performed in those environments.

As a person skilled in the art of medical imaging will recognize from the previous detailed description and from the figures and claims, modifications and changes can be made to the preferred embodiments of the invention without departing from the scope of this invention defined in the following claims.

I claim:

1. A method of representing a medical event, comprising the steps of:
(a) collecting 3D information on an event;
(b) identifying a non-linear aspect of the event;
(c) determining a non-planar slice of the 3D information that represents the non-linear aspect; and
(d) outputting the non-planar slice as a representation of the event.

2. The method of claim 1 wherein step (a) includes generating acoustic waves and receiving acoustic echoes.

3. The method of claim 1 wherein step (b) includes identifying a non-linear portion of a medical instrument.

4. The method of claim 3 wherein step (b) includes sensing one or more markers on the medical instrument.

5. The method of claim 4 wherein step (b) includes sensing a coating on the medical instrument.

6. The method of claim 1 wherein step (b) includes identifying a non-linear trajectory of a medical instrument.

7. The method of claim 6 wherein step (b) includes time-based pattern recognition techniques.

8. The method of claim 1 wherein step (b) includes identifying a non-linear trajectory of blood flow.

9. The method of claim 8 wherein step (b) includes time-based pattern recognition techniques.

10. The method of claim 1 wherein step (b) includes identifying a non-linear aspect of a medical target.

11. The method of claim 10 wherein step (b) includes pattern recognition and segmentation techniques.

12. The method of claim 10 wherein step (b) includes receiving a user selection of a segment.

13. The method of claim 1 wherein step (c) includes pattern recognition and segmentation techniques.

14. The method of claim 1 where in step (d) includes visually displaying the non-planar slice.

15. The method of claim 1 wherein step (b) includes identifying a second non-linear aspect of the event, and wherein step (c) includes determining at least one non-planar slice of the 3D information that represents the first non-linear aspect and the second non-linear aspect.

16. The method of claim 15 wherein step (c) includes determining two non-planar slices, and wherein step (d) includes outputting the two non-planar slices.

17. The method of claim 15 wherein step (b) includes identifying a non-linear portion of a medical instrument and a non-linear aspect of a medical target.

18. The method of claim 15 wherein step (b) includes identifying a non-linear trajectory of a medical instrument and a non-linear aspect of a medical target.
